(19) United States
(12) Patent Application Publication
(10) Pub. No.: US 2007/0026517 A1
(43) Pub. Date: Feb. 1, 2007

(54) Method and bioreactor for the cultivation and stimulation of three-dimensional, vitally and mechanically reistant cell transplants

(76) Inventors: Ronny Schulz, Leipzig (DE); Augustinus Bader, Leipzig (DE)

Correspondence Address: LERNER GREENBERG STEMER LLP, P O BOX 2480, HOLLYWOOD, FL, 33022-2480, US

(21) Appl. No.: 10/576,618

(22) PCT Filed: Oct. 19, 2004
(86) PCT No.: PCT/EP04/11788

§ 371 Date: Apr. 21, 2006

(51) Int. Cl. C12N 5/00

(52) U.S. Cl. 435/325

(57) ABSTRACT

A process and a bioreactor for the manufacturing of three-dimensional, vital and mechanically-resistant cell cultures that can be cultivated and stimulated within a short time of each other or simultaneously. The bioreactor has a basic body connected to a reactor lock so that it is pressure proof and sterile, this creating at least one reactor chamber, a storage space for a transplant and a mini actuator being implemented in this. The bioreactor is also equipped with at least two hose coupling connections for the feeding and discharging of the medium in addition to the gassing. The system enables GMP-conform transplant cultivation under guaranteed sterile conditions, of three-dimensional, vital and mechanically-resistant cell cultures, preferably cartilage-cell constructs which can hereby be cultivated and stimulated in a locked mini-bioreactor simultaneously, consecutively or within a time-controlled process according to GMP guidelines. These so-called transplants which are cultivated in this manner are then available as replacement tissue material for the therapy of connective and supporting tissue defects, direct joint traumas, rheumatism and degenerative joint disease, for example and can present an alternative to the conventional therapy approaches, such as micro fracturing or drill perforation in arthrosis of the knee joint, for example.

The invention relates to a process and an arrangement for the cultivation of three-dimensional, vital and mechanically-resistant cell cultures, preferably cartilage-cell constructs which can hereby be cultivated and stimulated in a locked mini-bioreactor simultaneously, consecutively or within a time-controlled process according to GMP guidelines. These transplants which are cultivated in this manner are then available as replacement tissue material for the therapy of connective and supporting tissue defects, direct joint traumas, rheumatism and degenerative joint disease, for example and can with an arthrosis of the knee joint present an alternative to the conventional (operative) therapy approaches, such as micro fracturing or drill perforation, for example.

With Tissue Engineering which above all concerns itself with the in-vitro reproduction of endogenic, so-called autologous cell material, one attempts to cultivate functional replacement cell and tissue structures which could be inserted into the defective tissue during a transplantation phase.

To this end, cell cultures (e.g. joint cartilage cells) are routinely reproduced in the laboratory. The actual reproduction of these cells (e.g. chondrocytes) takes place in a monolayer culture on the bottom of a coated cell culture flask or dish in accordance with standard protocols which also include the addition of tissue-related growth factors, mediators and inductors.

The objective of these additive factors is for example, the stimulation of the special ability which cartilage cells have to synthesise a sufficient number of extracellular matrix components (ECM), in order to achieve a mass ratio of 1% chondrocytes to 99% extracellular matrix components during the in-vitro reproduction, this being the ratio which exists in functional joint cartilage (Stockwell RA: The cell density of human articular and costal cartilage. J Anat. 1967; 101(4):753-763; Hamerman D, Schubert M: Diarthrodial joints, an essay. Amer J Med. 1962; 33:555-590). As this does not appear to be possible by simply adding medium supplements, an attempt is made to affect or stimulate these cartilage cells by applying various ways and means in order to render it possible to cultivate suitable replacement autologous (hyaline) cartilage with a high degree of differentiation in the laboratory.

The described reproduction of cell cultures and the cultivation of replacement tissue structures has numerous disadvantages.

This passive cultivation of cartilage cell cultures in a two-dimensional surface culture on a simple culture dish which is coated with a culture medium does not produce an active stimulation of the cartilage cells which are capable of differentiation.

From Minuth, W. W., Kloth S., Aigner J., Steiner P.: MINUSHEET-Perfusionskultur: Stimulierung eines gewebetypischen Milieus. Bioscope 1995; 4:20-25 a concept is known which attempts to avoid this disadvantage in that one places the cell material taken from the patient in an artificial carrier structure which has biophysical properties which are similar to those of the cartilage tissue and which permits a network-type connection between the multilayer arranged cells and which then carries out a perfusion cultivation in a suitable bioreactor. Numerous experiments show an increased cell differentiation capability as a result of an increase in synthesized ECM which results from this three-dimensional cultivation of chondrocytes in the most differentiated biocompatible and bio absorbable matrixes, e.g. the hydrogels, alginates, agaroses (Benya and Shaffer: Dedifferentiated chondrocytes reexpress the differentiated collagen phenotype when cultured in agarose gels. Cell. 1982; 30:215-224.) of various concentrations.

This spatial dimension which is thereby created therefore simulates the original ratios of the chondrocytes in living tissue such as in knee and hip joints, for example and therefore represents an advantageous adaptation of in-vivo situations.

With the adherent surface cultivation of the cells, the satisfactory supply of medium supplements is relatively simple as these cultures are situated immediately on or under the cells respectively, thereby permitting an unimpaired material exchange via diffusion.

Contrary to this, when using three-dimensional matrixes with imbedded cells in a static cultivation schema, it comes to the formation of concentration inclines or gradients which can limit the transportation of the material in medial construct regions, thereby having a negative effect on the optimal culture offer for the cell layers.

This impairment during the cultivation of cell material in spatial carrier matrix is counteracted by the induction of medium perfusion or transfusion through the construct.

This active process through this carrier structure ensures a homogenous nutrient supply in the cells and results in a continuous metabolite removal of the chondrocytes. In addition, the dynamic cultivation schema guarantees a higher gas entry and mechanically stimulates the cell layers subject to the selected medium perfusion flow resulting in a shearing force in μPa. (Raimondi, M. T., F. Boschetti, et al.: Mechanobiology of engineered cartilage cultured under a quantified fluid-dynamic environment. Biomechan Model Mechanobiol. 2002; 1:69-82)

An additional disadvantage with the reproduction of cells and a transplant results from the fact that the “cell culture flask” is not absolutely sterile. Even routine tasks such as changing the media, seeding the cell and even the harvesting of it result in a risk of the cell culture in it being infected, as the corresponding culture vessels have to be opened and the working in a laminar flow workbench does not enable the 100% sterility of the working environment within the meaning of the “Basic Rules of the World Health Organisation for the Manufacturing of Pharmaceutical Products and the Assurance of their Quality ” (Good Manufacturing Practice—WHO directive)to be guaranteed.

Furthermore, this passive system does not permit a maximum gas exchange through the diffusion-permeable cover and between the culture media and the cell layer on the bottom. In order to avoid these disadvantages of the culture flask, one has in recent years increasingly accelerated the development of automated, self-contained bioreactor systems for the generation of replacement tissue structures. They can then (Freed und Vunjak-Novakovic: Microgravity tissue engineering. In Vitro Cell Dev Biol Anim. 1997; 33:381-385) offer the advantage of sterile, controllable cultivation and stimulation of three-dimensional transplants. By combining the Tissue Engineering with the possibilities provided by process technology and biotechnology, the steering and control of selected cultivation parameters such as the gassing with CO₂ or O₂ respectively, temperature control, the exchanging of culture media, the taking of samples etc. in the bioreactor system are rendered possible. (Obradovic, Carrier, Vunjak-Novakovic and Freed: Gas exchange is essential for bioreactor cultivation of tissue engineered cartilage. Biotechnol Bioeng. 1999; 63:197-205).

When designing bioreactors, a well thought-out system must always be created, in which it is possible to regulate the processes by artificial means. When it comes to cultivating a particular tissue, the bioreactor system must be able to reproduce the physiological conditions and processes in-vivo as accurately as possible. All of the bioreactor systems work on the cultivated material with at least one kind of mechanical stimulation.

The lining of the positive features of a controlled bioreactor cultivation of autologous replacement tissue materials in a biogenous matrix under perfusion stimulation with a culture medium therefore represents the logical consequence of guaranteeing automated sterile or GMP-suitable transplant cultivation for the cultivation vital cartilage cells for example, with an increased ECM-synthesis performance.

A perfusion reactor is known from DE 4306661A1 and from Sittinger M, Bujia J, Minuth W W, Hammer C, Burmester G R: Engineering of cartilage tissue using bioresorbable polymer carriers in perfusion culture. Biomaterials. 1994; 15(6):451-456, by which the cells are embedded in a polymer layer and is additionally encased in an agerose capsule. An artificial culture media flows through the cylindrical glass reactor with a flow rate of 0.016 ml/min. The reactor itself is situated in a corresponding tissue incubator with standardised conditions. Sterile filters on the culture medium depot enable a gas exchange to take place with the outside environment.

Continuative experiments carried out with this type of reactor by Bujia J, Rotter N, Minuth W, Burmester G, Hammer C, Sittinger M: Cultivation of human cartilage tissue in a 3-dimensional perfusion culture chamber: characterization of collagen synthesis. Laryngorhinootologie. 1995; 74(9): 559-563 und Kreklau B, Sittinger M, Mensing M B, Voigt C, Berger G, Burmester G R, Rahmanzadeh R, Gross U: Tissue engineering of biphasic joint cartilage transplants. Biomaterials.

1999; 20(18):1743-1749 used co-polymer tissues of vicryldiaxonon layers and polydioxanon layers, which have been soaked in Poly-L-Lysine or collagen fibres of type II. Human chondrocytes are imbedded in these layers and cultivated under perfusion for a period of two weeks. Under use of a two-phase model of a co-polymer, one polyglycolic acid and a poly-L-lactic acids (Ethicon), which was attached to a calcium-carbonate product, the period was extended to 70 days.

An additional system which is very similar to the above perfusion bioreactor was constructed by Mizuno S, Allemann F, Glowacki J: Effects of medium perfusion on matrix production by bovine chondrocytes in three-dimensional collagen sponges. J Biomed Mater Res. 2001; 56(3):368-375. Contrary to the reactor which has already been described, this has a closed area for the artificial culture media. The main part of the cultivated material is situated in a cylindrical glass column which is 1 cm wide and 10 cm long. The column is filled with numerous cell/polymer frameworks, each having a size of 7×15 mm, these not being additionally encapsulated. The artificial culture medium is led from a depot through the column and the complete system at a speed of 300 μl/min. This system was used to examine bovine chondrocyte frames in collagen sponges with regard to their reaction to perfusion during a cultivation period of 15 days.

A bioreactor device is also known from the U.S. Pat. No. 5,928,945 in which the adherent cartilage cells are subjected to defined flows or shearing force in a growth chamber which resulted in the detection of an increased collagen type II synthesis.

Parallel to the development of perfusion bioreactors, research groups concerned themselves with the design of bioreactors which exercise diverse mechanical load processes in explants, cell samples or cell/polymer frames. When constructing bioreactors for the stimulation of cartilage cells, their design orients itself to the implementation of mechanical plungers, etc, as these apply uniaxial pressure to cartilage transplants in order to imitate the most important form of load applied to human cartilage tissue. Many of these pressure systems have great design similarities.

The pressure chamber of a system developed by Steinmeyer J, Torzilli PA, Burton-Wurster N, Lust G: A new pressure chamber to study the biosynthetic response of articular cartilage to mechanical loading. Res Exp Med (Berl). 1993, 193(3):137-142 comprising a titanium housing which is coated by a polyethylene layer on the inside. The experiment sample with a maximum diameter of 10 mm can be placed on the floor of the chamber and covered with around 7 ml of an artificial culture media. As the model does not have an artificial culture medium perfusion system, only pressure generations in phases with short cultivation times are possible. The load system which exercises the corresponding pressure on the experiment sample comprises a porous pressure crucible which leads through the chamber lock and is either moved by means of simple weights or an air cylinder with pressure cylinder which is mounted above the chamber.

The system published by Lee D A, Bader D L: Compressive strains at physiological frequencies influence the metabolism of chondrocytes seeded in agarose. J Orthop Res. 1997; 15(2):181-188 which is set in motion by a drive is capable of being able to exercise pressure on 24 test samples simultaneously. The drive is mounted on a frame which leads around the incubator and transfers the force down to the loading plate inside the sterile box. The steel loading plate has 24 steel bolts with a Plexiglas indentation with a diameter of 11 mm. The drive provides various loads which depend on the degree of deformation. This system is used for the cultivation of bovine chondrocyte/agarose frames for a period of two days. Static and additional cyclic loads (0.3-3 Hz) with a maximum tension amplitude of 15% are generated.

The disadvantage of numerous pressure stimulation reactors is that the cell culture constructs cannot be perfused with a culture media during a pressure load so that the effect of a multiple cell stimulation cannot be tested. Furthermore, this lack of culture supply is opposed by an optimal metabolism exchange and the maximum synthesis of extracellular matrix components in cartilage cells, for example.

Pressure and perfusion systems such as those which are described in the U.S. Pat. No. 6,060,306 and the DE-patent 198 08 055 enable a simultaneous multiple stimulation with parameters such as perfusion flow, the resulting induced shearing forces and an uniaxial pressure load.

The disadvantage of reactors which enable a pressure stimulation to take place is above all that they necessitate the entering of the bioreactor space which preferentially contains an autologous transplant by pressure mediators, mainly plungers and pistons, etc. which are driven by servomotors, or similar and that a defined pressure load is then applied to the cell construct. The insertion of these pressure applicators into the sterile system renders the designing of self-contained pressure application reactors extremely difficult so that these systems are of an increased complexity. A usage of the (potentially non-sterile) systems is therefore only given in basic research as an application of these devices and process in the medical sector contradicts parts of the directives in the existing Medical Preparations Act.

All of the bioreactor apparatuses used for the cultivation and stimulation of replacement autologous tissue structures therefore serve the WHO Good Manufacturing Practice Directive (“Basic Rules for the Manufacturing of Pharmaceutical Products and the Securing of their Quality”) and the German Pharmaceuticals Act (Arzneimittelgesetz) (AMG), the “Pharmaceutical Inspections Convention” and GMP-Directive 91/356/EEC. The risk of an infection or the impossibility of it being possible to fully guarantee the sterility of the systems therefore constitutes no grounds for the issuing of a manufacturing license pursuant to Section 13 AMG.

The task of the invention is the creation of a process and a bioreactor for the manufacturing of three-dimensional, vital and mechanically-resistant cell cultures, by which they can be cultivated and stimulated within a short time of each other or simultaneously. The bioreactor should permit GMP-conform transplant cultivation under guaranteed sterile conditions.

The invention fulfils the task with the process described in Claim 1 and the bioreactor described in Claim 13. Advantageous forms of the process are described in Claims 2-12; Claims 14-57 describe other forms of the bioreactor.

The invented process and the invented bioreactor combines the cultivation and stimulation of GMP-conform manufactured, three-dimensional vital and mechanically-resistant cell cultures, preferably cartilage cell constructs, in a single reactor. Hereby, the stimulation and cultivation can take place simultaneously, consecutively or in accordance with a time-controlled process. The transplants cultivated in this manner are available as replacement tissue material for the therapy of connective and supporting tissue defects, direct joint traumas, rheumatism and degenerative joint disease, for example.

The fundamental characteristic feature of the invented process and the invented bioreactor is that a transplant is in a self-contained reactor chamber which can be subjected to in-vivo-adaptive stimulus in many regards. This includes the perfusion of the spatial culture construct with a conditioned culture media which evokes organotypical shearing forces on the cell membranes and additionally permit an increased metabolic exchange to take place. A magnetic, piston-like pressure stamp which acts as a load applicator to the cell culture is situated above the transplant in this self-contained bioreactor. The stamp is controlled by the bioreactor chamber in a contactless form, the tissue transplant being subjected to directed uniaxial pressure stimulation.. The contactless controlling of the mini-actuator is carried out by externally arranged control magnets whose directed (electro-)magnetic filed brings about a change of the stamp position within the bioreactor, resulting in an organotypical dynamic or static pressure stimulation, respectively.

The process and the bioreactor have the advantage which has already been described that a stimulation of the cell cultures can also take place during cultivation. The cultivation or regeneration of connective and supporting tissue structures and functional tissue systems (cartilage, bones, etc.) are especially possible.

When used in a sterile process, the apparatus enables cell transplants to be cultivated which are characterised in that they are especially synchronously perfused and pressure-loaded, this resulting in an increased production of matrix components (e.g. cartilage cell cultures). Due to its degree of automation, this device minimises the number of stages, thereby reducing the risk of infecting the cell culture. The automated cultivation and stimulation of the transplants also guarantees defined and reproducible process cycles. Due to the design characteristics of the invented bioreactor, a self-contained bioreactor circulation is guaranteed and this therefore enables a stringent autologous cultivation or stimulation of replacement tissue structures under adherence to the GMP-directives.

An additional field of use of the bioreactor is the pharmaceutical active ingredient testing for the characterization of proliferation and differentiation-relevant ingredients or ingredient combinations on transplants.

An explanation of the invented process and the invented bioreactor now follows execution examples. The corresponding illustrations show:

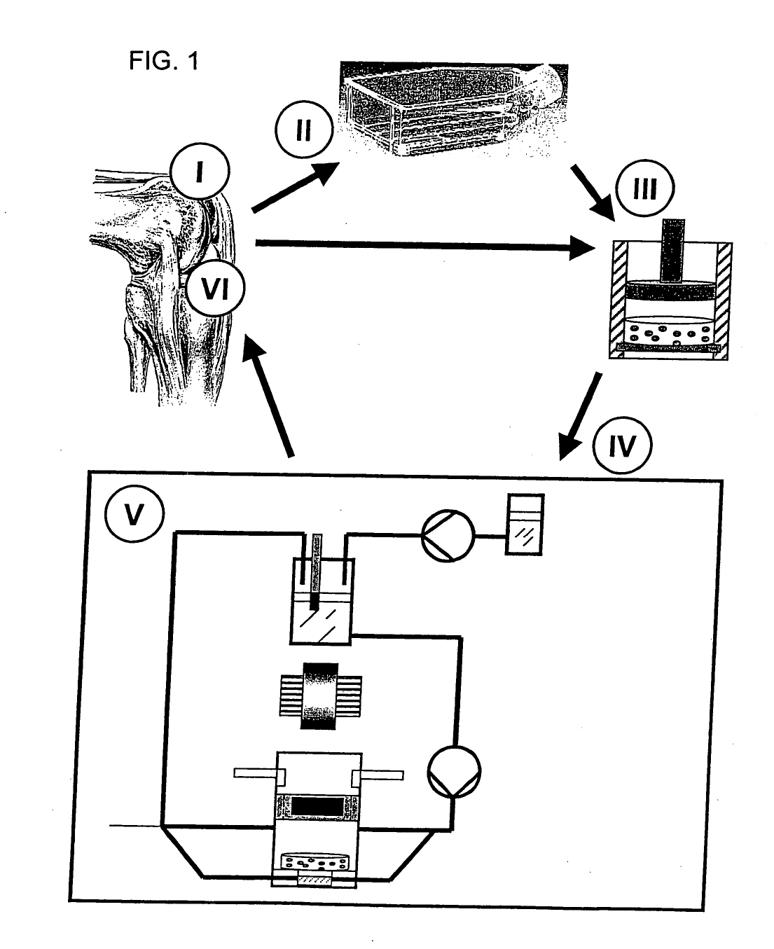

FIG. 1: Process for manufacturing transplants

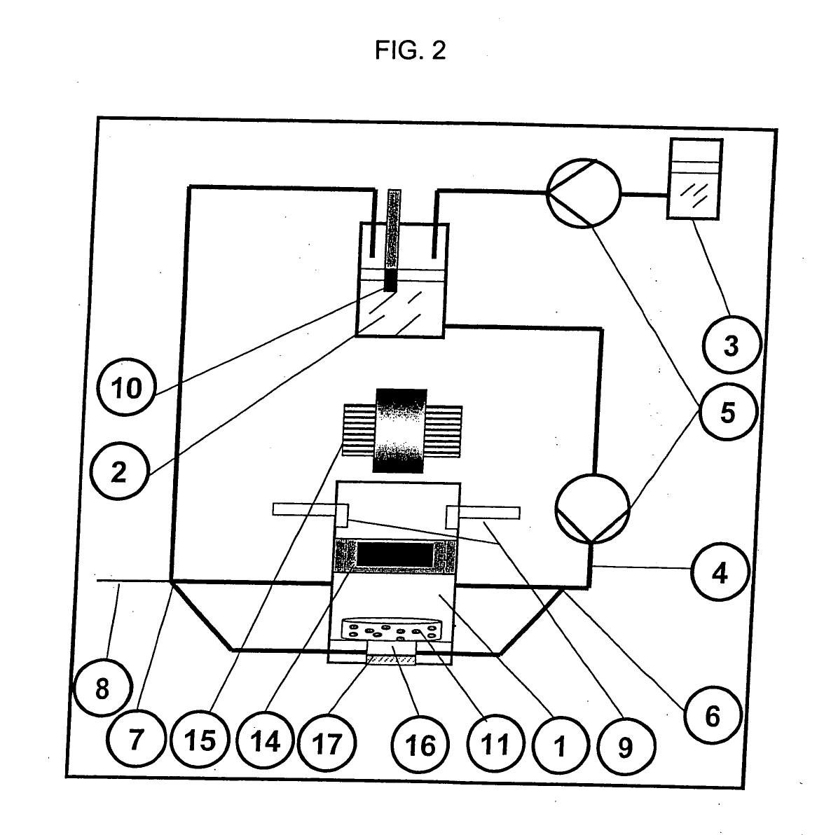

FIG. 2: GMP-Bioreactor system schema

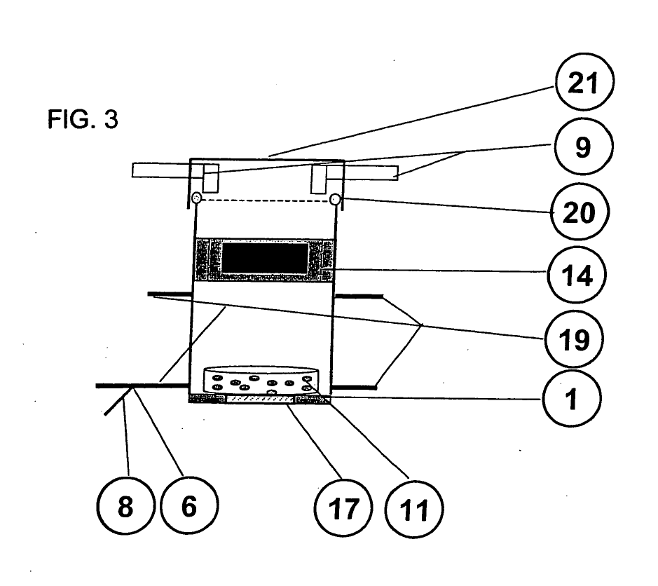

FIG. 3: Single-chamber bioreactor schema

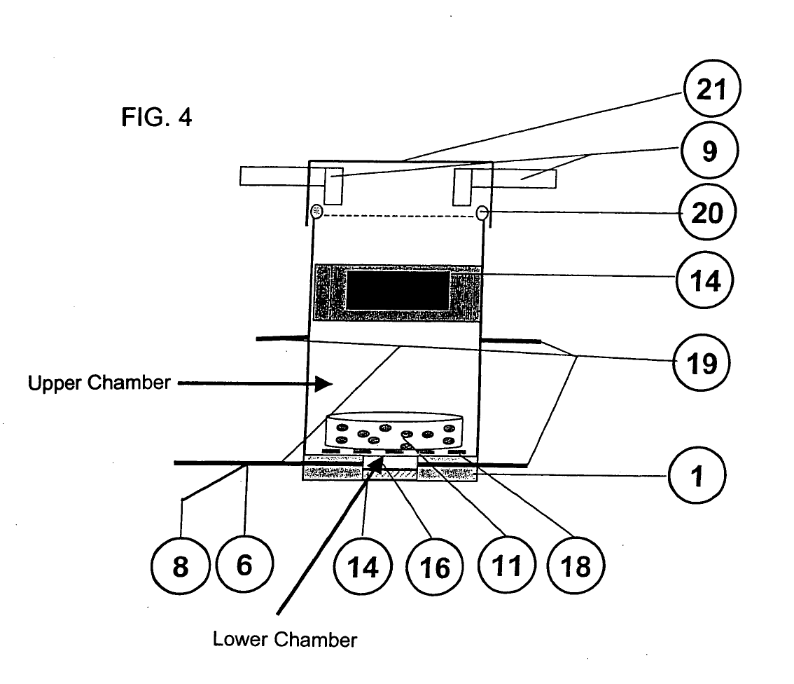

FIG. 4: Double-chamber bioreactor schema

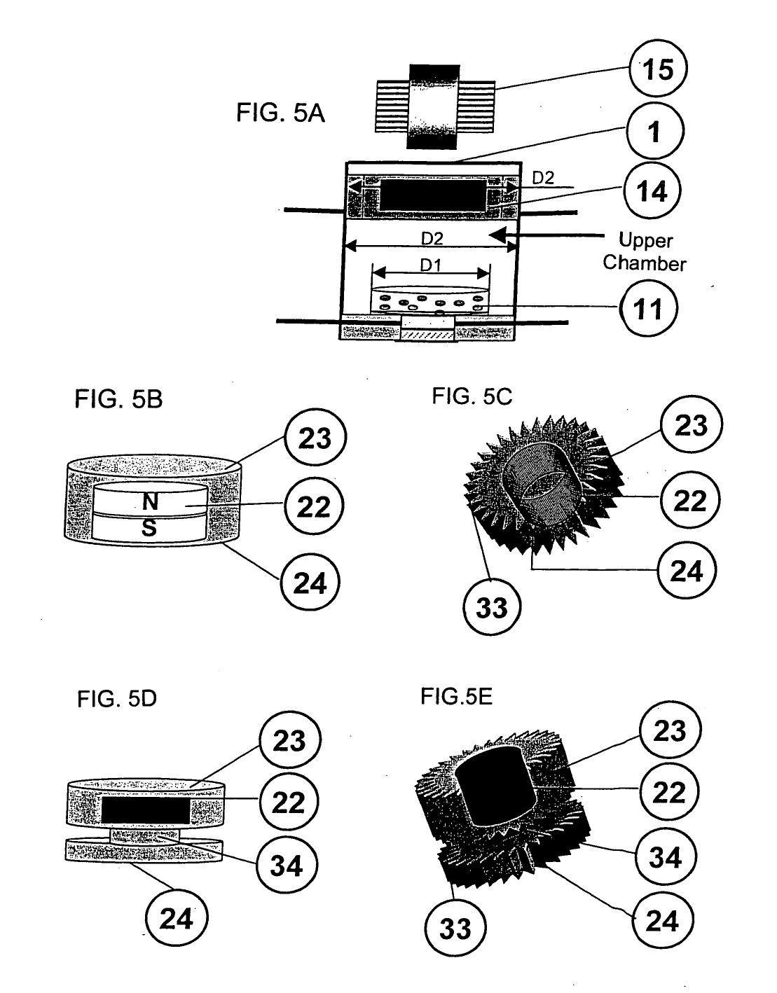

FIG. 5: Design and form of executions of the mini actuator

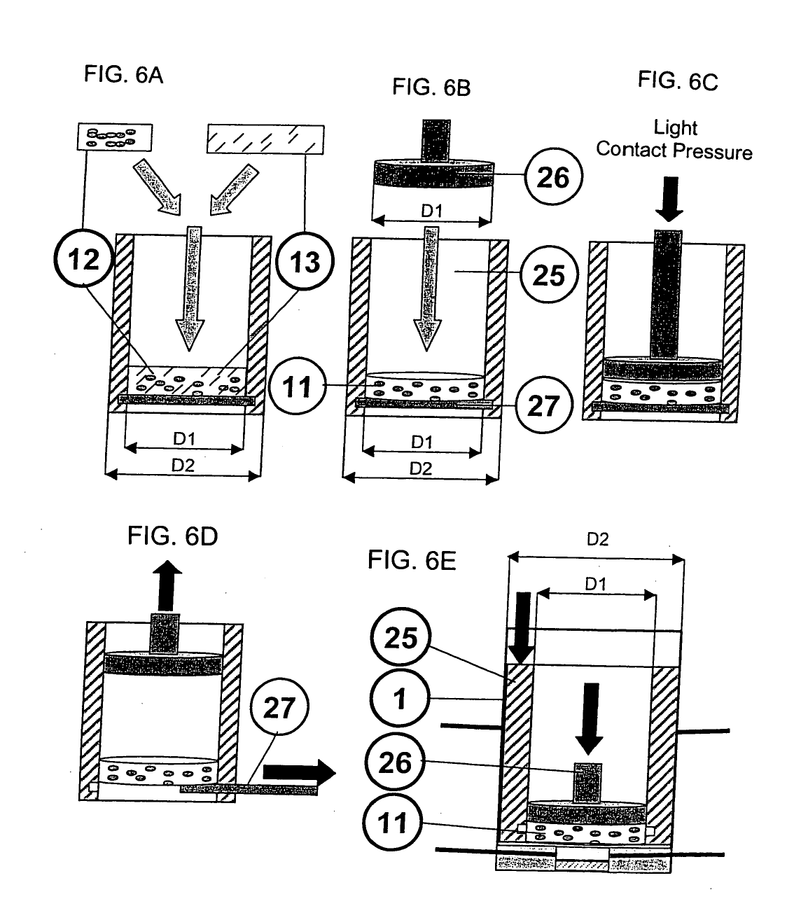

FIG. 6: Schema showing the construct manufacture and seeding of the construct in the bioreactor

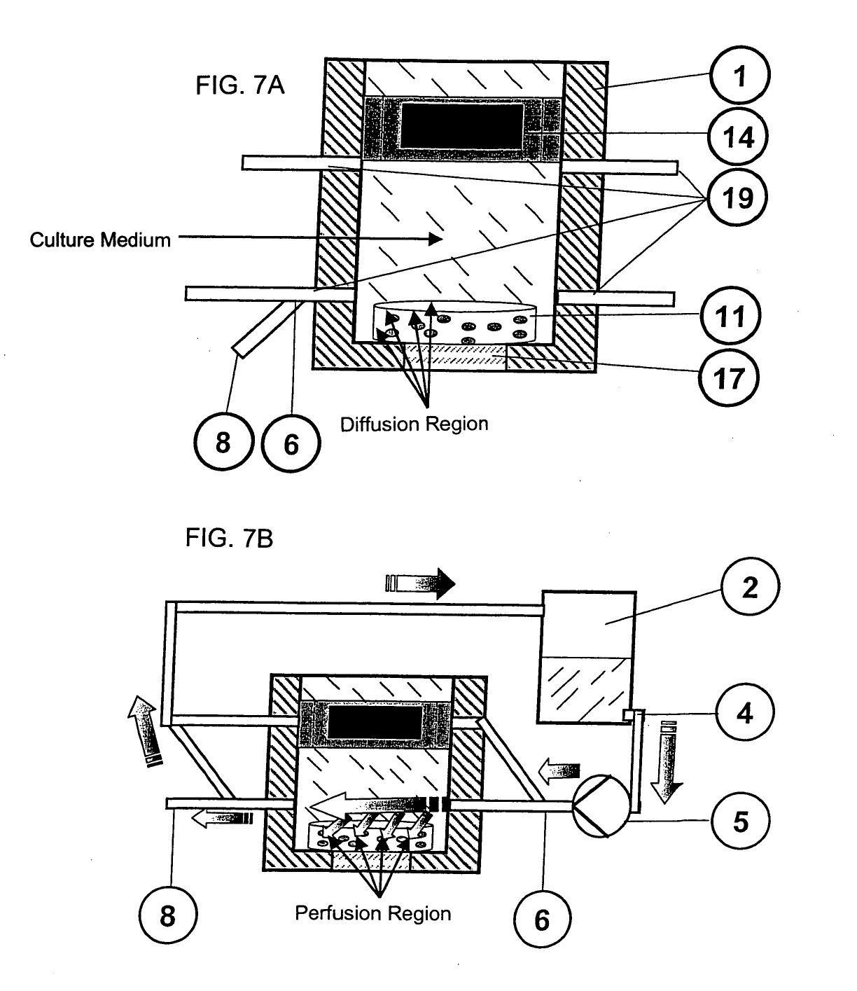

FIG. 7: Schema showing the technical equipment for construct perfusion and media blending in a single-chamber reactor

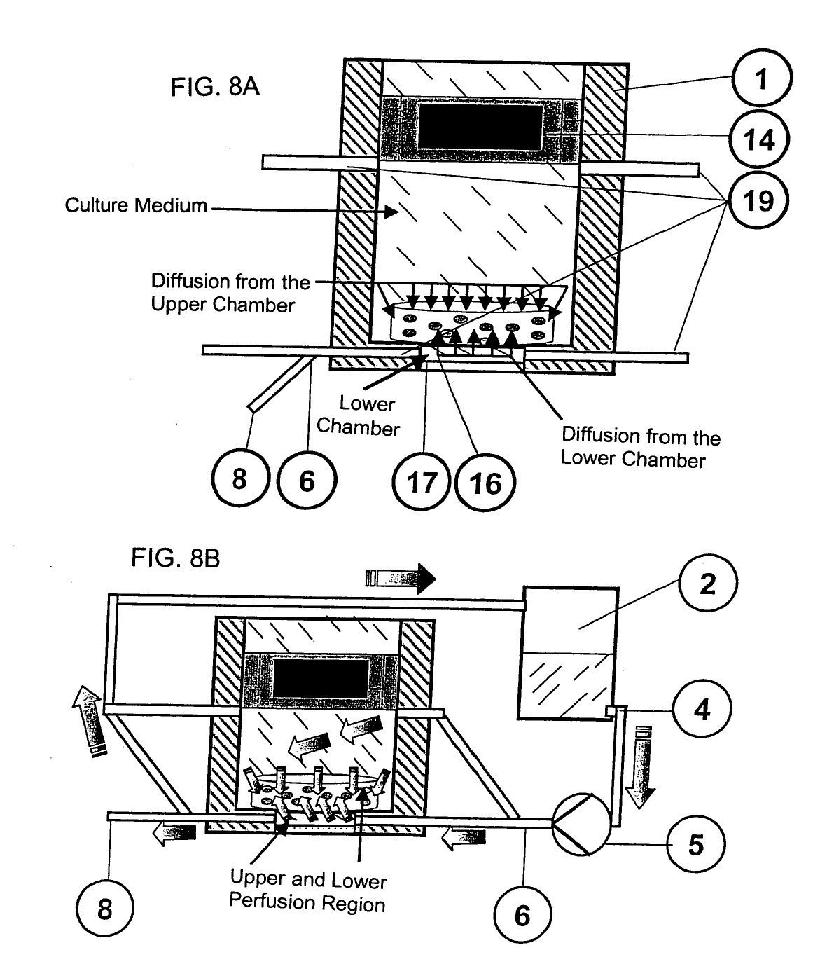

FIG. 8: Schema showing the technical equipment for construct perfusion and media blending in a double-chamber reactor

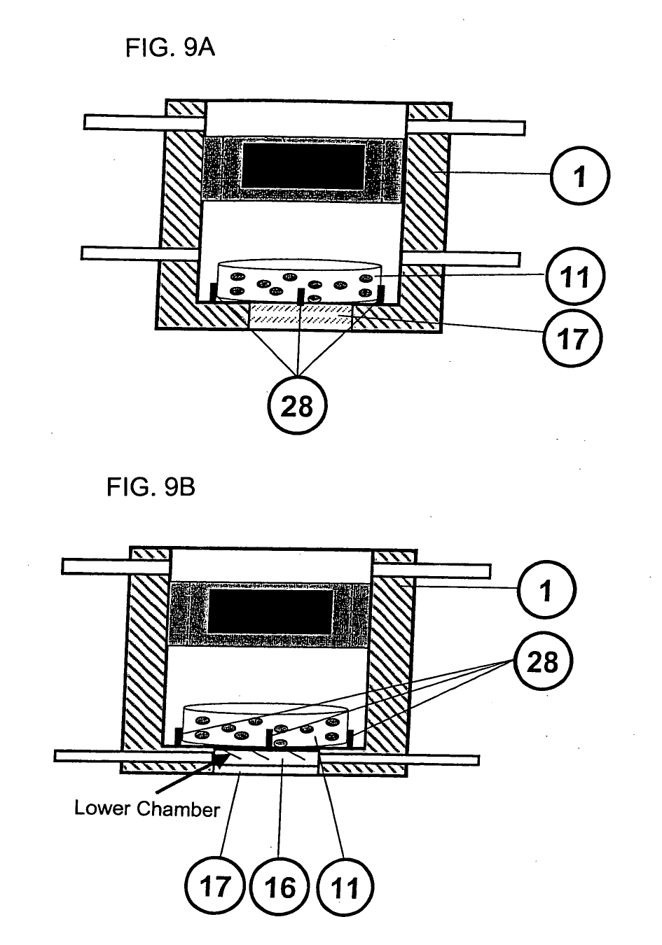

FIG. 9: Schema showing the fixation of the transplant in the bioreactor

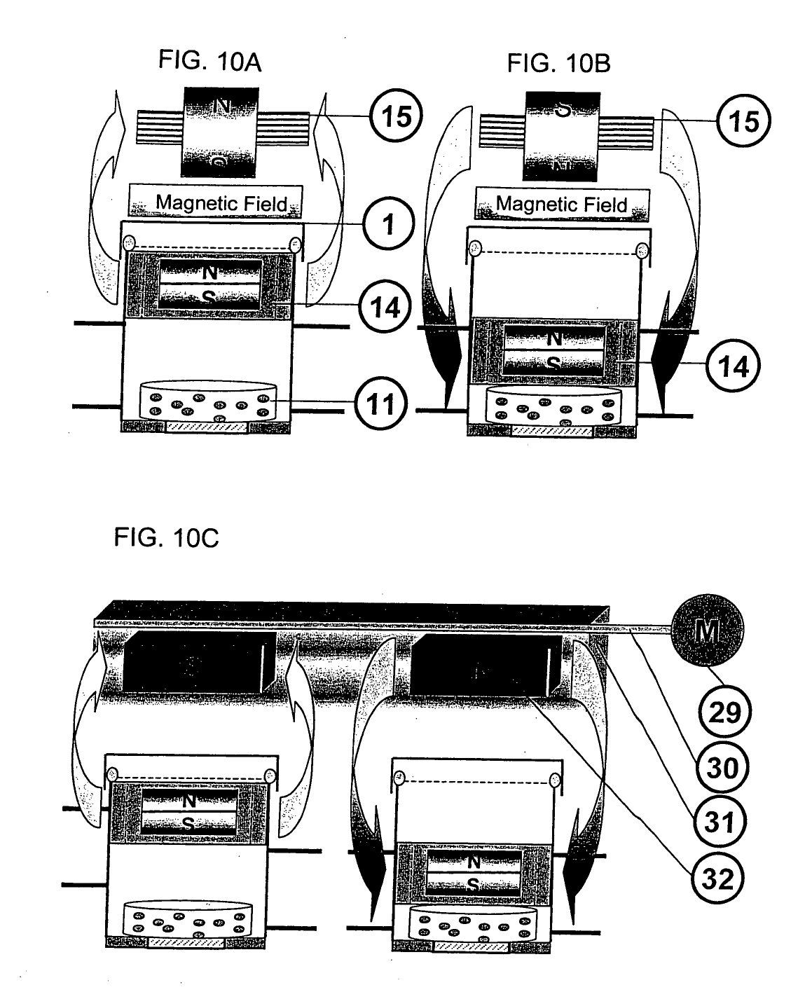

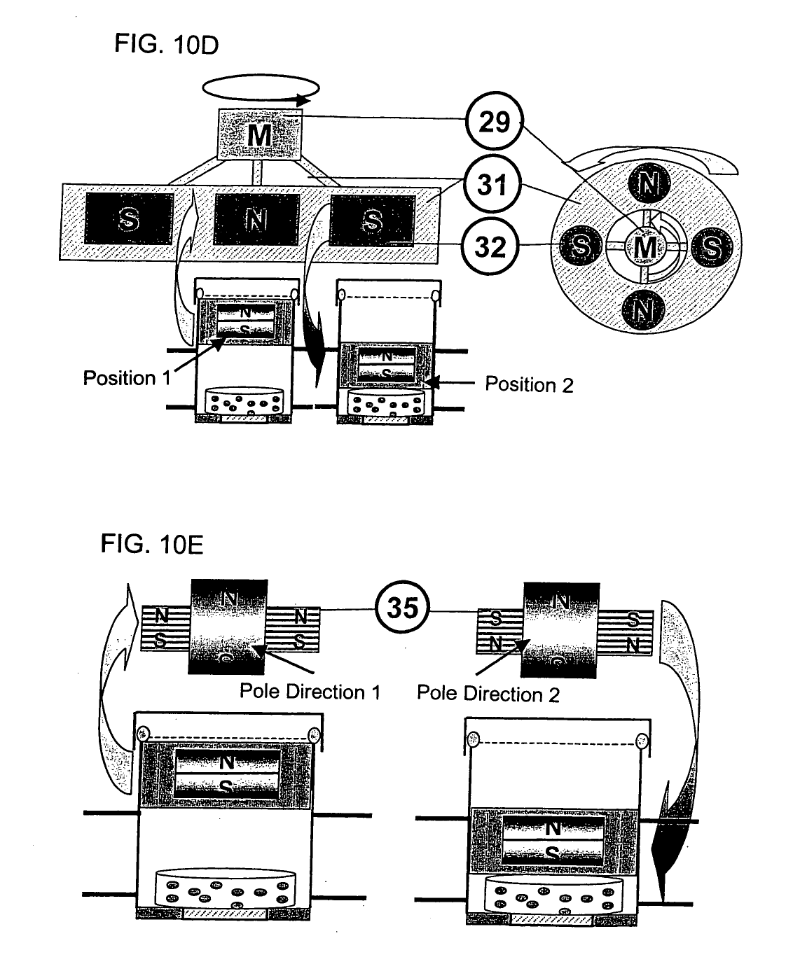

FIG. 10: Magnet systems for controlling the mini actuator

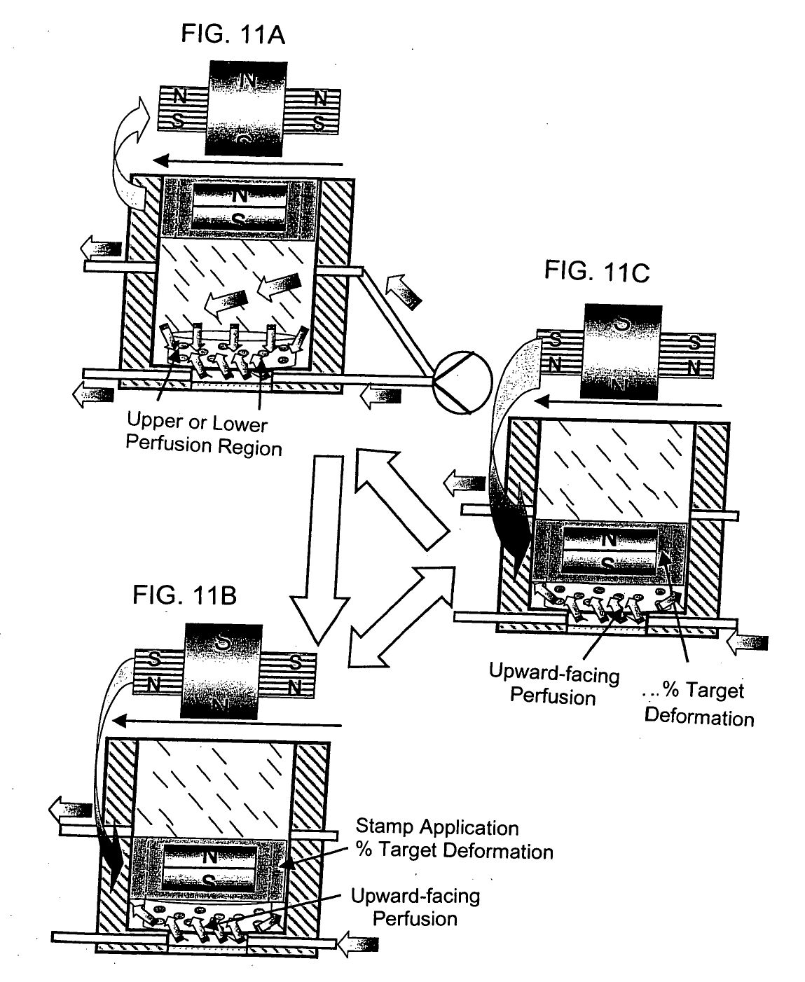

FIG. 11: Schema showing the stimulation in the double-chamber reactor

### EXAMPLE 1

Process for Manufacturing Transplants

FIG. 1 shows the use of the bioreactor for the synchronous cultivation and stimulation of three-dimensional cell transplants, taking the cartilage tissue transplantation as an example.

To this end, the patient first of all (I) as healthy cell material (e.g. articular cartilage) and blood taken from him by minimal invasive means. The attained cells are separated and counted under enzymatic digestion, they then being either sown out in monolayer flasks (II) according to standard tissue engineering methods, where they multiply in a stringently analogous manner or they are immediately used for the manufacturing of the construct (III). Hereby, the cells are added top a three-dimensional transplant structure of biocompatible or absorbable carrier materials (e.g. hydrogels, agaroses, collagens, hydroxylapatites, polymer complexes etc.). The suspended cells (e.g. chondrocytes) are mixed with the biogenic support structure (e.g. agarose), placed in a seeding piston and hardened into a cylindrical transplant form, for example (e.g. cartilage-agarose-matrix). This in-vivo adaptive, three-dimensional structure especially results in a (re-)differentiation and resulting synthesis of tissue-typical substances and matrix components (e.g. collagens, proteoglycane)in connective and supporting tissue cells (e.g. chondrocytes).

This seeding piston with the spatial cell transplant inside it is inserted into the bioreactor (IV), the transplant then being pressed out and positioned in the bioreactor. The GMP-suitable cultivation and stimulation of this cell construct takes place in the newly developed bioreactor apparatus (V) GMP-conform simultaneously, consecutively or time-controlled. During this phase, the cell transplant can be caused to create an increased differentiation and expression of organotypical markers (stimuli such as shearing force, perfusion, deformation, mechanical load) by means of this multiple in-vivo-similar stimulation.

A highly-vital, matrix-rich cell culture construct regenerates in the bioreactor after a short amount of time. This autologous transplant is removed (VI), adapted to the geometry of the tissue defect if necessary and subsequently transplanted into the defective connective or supporting tissue.

### EXAMPLE 2

Bioreactor System Schema

FIG. 2 illustrates a form of execution of the bioreactor system (with the double-chamber bioreactor) for the autologous cultivation and multiple stimulation of cell transplants in a self-contained reactor structure with a GMP-conform process method.

In this execution example, the complete equipment for guaranteeing an optimal temperature, air humidity and composition is situated in a temperature-controlled and gas-regulated incubator. A separate design is also possible, in that the bioreactor 1 and the medium are situated in the incubator, the other technical components being situated outside the incubator.

The bioreactor 1 itself and the components used therein are biologically and chemically inert and can be treated by autoclave. Furthermore, the bioreactor carcass and the screw-on cover are of materials which are either non-magnetic (e.g. synthetic materials) or weak-magnetic (e.g. vanadium-4-steel).

The culture medium is fed to the bioreactor 1 after being taken from the medium reservoir 2 and passing through the hose system 4 with the 3-way valve 6 and the 4-way valve 7 by means of the circulation pump 5. This culture medium can be enriched with autologous additive factors taken from the supplement reservoir 3 (growth factors, mediators, etc.) which were obtained from the patient's blood. The medium is added to bioreactor 1 and therefore the transplant 11 in a batch, fed-batch or continuous process.

When the circulation is closed, the medium then enters the medium reservoir 2 via hose system 4, the reservoir being equipped with measuring probes for controlling the physicochemical parameters, e.g. pH, pCO₂ and pO₂. If the medium is seen to be used, it can be drained off into an external locked waste vessel via hose system 4. In both cases there is the possibility of deviating a sterile medium sample from the reactor circulation to a sample taking section 8 via the valve device 7 for further analysis.

The transplant 11 which is to be cultivated and stimulated is positioned in a medial position on the bottom of the reactor. A second, smaller chamber can be situated underneath the transplant 11. The flow space is supplied with the media via the hose system 4 and can be filled with a strongly porous but thin sinter material 16. This lower chamber can be sealed off by a thin sheet of transparent glass 17 and serve as a microscopy opening for inverse microscopes.

In addition to the biosensors 9 which are inserted in the bioreactor cover, the upper chamber of the bioreactor 1 also includes the mini actuator 14. This mini actuator 14 which is designed as a magnetic stamp serves as a contactless pressure applicator and is controlled by the control magnets or the coil 15.

### EXAMPLE 3

Single-chamber Bioreactor Schema

FIG. 3 shows a possible form of execution of the bioreactor 1 comprising a culture chamber which serves the implementation of the contactless controllable mini actuator 14.

The bioreactor 1 which is designed as a single-chamber bioreactor comprises a carcass and the bioreactor lock 21 which is additionally sealed by a pinch ring 20. Biosensors 9 are integrated in the cover construction which serves to take on-line measurements of glucose and lactate concentrations, among others. An exactly fitting integrated mini actuator 14 is situated above the transplant 11 in the reactor chamber, the transplant resting on a special reactor floor with an inserted transparent glass plate 17.

For the supplying of the transplant 11 with the medium, a minimum of one feed and one discharge penetrate the bioreactor 1 via Luer connectors 19. A sample taking section 8 is integrated at least one of the discharges 19 via a 3-way valve 6.

### EXAMPLE 4

Double-chamber Reactor Schema

FIG. 4 shows an additional form of execution of a bioreactor 1 comprising two chambers, whereby the upper comprises the pressure stamp 14, the lower serving the flowing against underneath the transplant 11. The function, character and requirement of the components 1, 6, 8, 9, 14, 19-21 in this form of execution do not differ from those in the bioreactor 1 described in example 3.

At least one feed and one discharge 19 are integrated in the upper and lower reactor chambers in order to achieve a valve-controlled flowing against in the individual chamber and the transplant 11.

The dimension of the lower chamber is of a diameter which is smaller than that of the transplant 11. This chamber includes a flat exact-matching plate of a porous sinter material 16 which enables an inverse microscopy to be carried out through the flush glass plate 17 and the membrane 18 to the transplant 11 without impairment. This plate of a sintered material 16 in the lower reactor chamber has an additional important function in this apparatus. When the transplant 11 is subjected to mechanical load by the pressure stamp 14, it prevents an undesirable pressing of the gel-like cell construct 11 into the chamber space. Depending on the user's support matrix and its viscosity, the use of a fluid-permeable membrane 18 between the sinter material 16 and the transplant 11 is intended in order to avoid a blending of the carrier material with the sinter material 16.

### EXAMPLE 5

Design and Form of Execution of the Mini Actuator 14

FIG. 5 shows the design, geometry and different forms of the mini actuator 14 which slides into the reactor chamber it hereby having a perfect fit (shown here in a double-chamber model as an example), it here asserting axial pressure forces on the transplant 11 which is lying on the floor of the reactor.

This magnetic pressure applicator 14 is controlled in its vertical position in the bioreactor 1 contactless by means of externally arranged control magnets 15 in accordance with the invention (see FIG. 5a). An absolutely vertical compression can be ensured on the one hand by medially positioning the transplant 11 in the bioreactor 1. On the other, an exact fit dimensioning of the pressure stamp diameter D2 to the internal bioreactor D2 must also take place. This enables the mini actuator 14 to be inserted into the bioreactor 1 without the stamp jamming or inclining. In all bioreactor models, this diameter D2 is to be dimensioned larger than the external diameter D1 of the transplant 11.

FIG. 5b shows the characteristic design of this pressure unit 14. It has an extremely powerful permanent magnet 2, preferably of a neodymium-iron-boron compound which, upon the existence of the slightest magnetic and electromagnetic fields moves in the direction of the corresponding field. This permanent magnet 22 is of a varnished or galvanized form and encapsulated in a biological inert synthetic material—the enveloping body 23—. This preferably cylindrical enveloping body 23 with its exactly fitting external diameter slides into the bioreactor cylinder with low friction and exactly vertically. The underside of the plastic enveloping body 23 can-in addition to a level surface, have other organotypical negative forms as a stamp surface 24 impressed on it, so as to reproduce in-vivo adaptive positive forms (including curves, arches, etc.).

The novel actuator geometry which exists here without any flow channels 33 in the enveloping body 23, also provides a pump function resulting from a cyclical magnetic field generation. An upward movement of the mini activator 14 enables medium to be sucked into the reactor chamber as a result of the pressure and valve rations which exist in the bioreactor 1. A downward movement or pressure compression ion the transplant 11 results in this medium being pressed out of the bioreactor 1.

FIG. 5c shows an additional example of a form of execution of the mini actuator 14 which also includes a strong permanent magnet 22 and an enveloping body 23 with an individual stamp surface 24. This model has so-called flow channels 33 at the edge of its enveloping body 23 for flow optimization. This enables a medium flow of the mini actuator 14 to be carried out in the bioreactor chamber, so that less positioning force is required to overcome the media resistance. The enveloping body 23 must have at least 3 guide projections with an exactly matching external diameter D2 in order to ensure a planar positioning of the complete mini actuator 14 on the transplant 11.

FIG. 5d shows a modified pressure stamp 14 which is based on FIG. 5b but which has an extension nosepiece 34′, designed to create a spatial distance between the permanent magnet 22 and the cell culture-construct 11. The cause of this distancing of the permanent magnet 22 in the upper cylinder head from the transplant 11 is the minimisation of any field influences on the cell cultures 11.

FIG. 5e shows a mini actuator 14 based on FIG. 5d which has at least 3 flow channels 33 and 3 guide projections with an outside diameter D2.

### EXAMPLE 6

Schema Showing the Construct Manufacture and Seeding of the Construct in the Bioreactor 1

FIG. 6 shows the process and the equipment for manufacturing and seeding three-dimensional, preferably cylindrical cell matrix constructs.

In FIG. 6a (cell matrix seeding) multiplied (see FIG. 1, II) or freshly isolated (see FIG. 1, III) and prepared cells 12 are mixed with the biogen carrier structure 13, suspended to homogeneity and injected into the seeding piston 25. The exactly fitting seeding piston 25 has an internal diameter Dl which corresponds to the future external diameter of the transplant 11 and an external diameter D2 which corresponds to an internal diameter of the bioreactor 1.

FIG. 6b (stamp insert) shows the stamp insert 26 in the seeding piston 25. The exactly fitting planar stamp 26 with the outside diameter D1 is inserted in the hollow piston cylinder on the level sliding plate 27 during the hardening out or polymerisation of the corresponding cell matrix in the reactor piston 25.

The underside of this stamp 26 can be embossed with organotypical structures analogous to the stamp surfaces 24 of the mini actuator 14.

FIG. 6c (stamp application) shows the application of the stamp 26 on the transplant 11 in the seeding piston 25. The stamp 26 is placed on the cell frame with a slight assertion of application pressure in order to counteract a meniscus formation or curving of the upper side of the matrix of the transplant 11, in order to obtain a cylindrical transplant form, etc.

If an in-vivo adaptive surface is to be impressed on the transplant 11, this stamp application 26 must take place during the hardening out or polymerization phase respectively.

In FIG. 6d (removing the sliding plate), the applied stamp 26 is raised after the forming of the transplant 11 and the sliding plate 27 which should be preferably hydrophobic and is situated at the bottom of the seed piston is removed. In order to prevent a gel-type cell construct 11 adhering to the sliding plate 27 and the seeding piston, and inert foil or inert polymer fleece for example are used to line the surface.

FIG. 6e (construct seeding in the bioreactor) shows the seed of a cylindrical construct, taking the double-chamber bioreactor as an example. Hereby, the exactly matching seeding piston 25 is implemented in the bioreactor 1, the cell construct then being positioned medially in the prepared reactor by mean of the pressure stamp 26, the seeding device then being removed from the bioreactor 1. This prepared bioreactor 1 contains the porous sinter material 16 and a diffusion-permeable membrane 18, if required.

### EXAMPLE 7

Schema Showing the Technical Equipment for Construct Perfusion and Media Blending in a Single-chamber Bioreactor

FIG. 7 shows the design and construction of the single-chamber reactor carcass and its effect on the diffusion and perfusion in transplant 11.

In the form of execution shown in FIG. 7a, four feeds and discharges with an integrated Luer connector 19 run into the bioreactor 1. Both their locations and positions can differ in order to optimize the flow, this therefore meaning that they can also enter the bioreactor carcass tangentially. A minimum of two feeds or discharges respectively enter the bioreactor 1. A sample taking section 8 can be installed at each discharging Luer connection 19 by means of a 3-way valve 6, for example.

A static cultivation method in the bioreactor especially results in a diffusion of the media in the upper and side edge areas of the cylindrical tissue transplant 11, for example and provides the cell culture with nutrients, among others whilst simultaneously transporting metabolic end products from the carrier matrix.

FIG. 7b shows a continuous feed of the culture medium from the medium reservoir 2 with the optional supplement reservoir 3 (not shown) behind it. The culture medium enters the bioreactor 1 through a minimum of one feed 19 after passing through the hose system 4 by means of a circulation pump 5 which is capable of apportioning.

The medium is discharged via a minimum of one discharge 19, where it enters the hose system 4 which enables a separate sample taking section 8 to be integrated at least one position by means of a 3-way valve 6.

The used medium can remain in the circulation as shown here, in that it enters the medium reservoir 2, from where it is extracted for a repeated continuous perfusion of the transplant 11. It can also be completely removed from the circulation. The transplant 11 is then cultivated by means of a batch or fed-batch process respectively.

A targeted continual feeding of the culture medium into the reactor chamber can result in a clear approach and through flowing of the transplant 11, when compared with the static schema shown in FIG. 7a. The induced perfusion results in deeper construct regions being thoroughly rinsed with the medium. This results in an optimization of the material exchange and in turn, an increased cell differentiation. This version of the construct approach flow exercises shearing force stimulation on the embedded cells.

### EXAMPLE 8

Schema Showing the Technical Equipment for Construct Perfusion and Media Blending in a Double-chamber Reactor

FIG. 8 shows a double-chamber bioreactor which permits an optimized flow, diffusion and perfusion of the transplant, thereby helping to improve the quality of the replacement tissue.

A version with static cultivation and diffusion is shown in FIG. 8a. The feeds or discharges 19 respectively which run into the bioreactor 1 number two as a minimum, whereby at least one of them must run into the lower and the upper reactor chamber. The positions, locations and densities of the two feeds or discharges 19 shown here for each chamber can differ in order to achieve a flow optimization.

The sample taking section 8 can be connected to any of the discharge oriented Luer connections 19 in both of the chambers by means of a 3-way valve 6, or similar.

In addition to a media diffusion of he upper and side transplant areas, the chamber in this design which have been set-up for the first time results in a diffusion of the culture medium from the porous sinter material in the region close to the floor of the carrier structure, the diffusion being underneath the transplant 11 during the static cultivation, this resulting in an improved metabolism throughout the transplant 11.

FIG. 8b shows a continuous feed of the culture medium from the medium reservoir 2 with the optional supplement reservoir 3 (not shown) behind it. The culture medium enters the upper and lower chambers of the bioreactor 1 through a minimum of one feed 19 after passing thorough the hose system 4 by means of a circulation pump 5 which is capable of apportioning.

The medium is discharged via a minimum of one discharge 19 per chamber, where it enters the hose system 4 which enables a separate sample taking section 8 to be integrated at least one position by means of a 3-way valve 6.

The used medium can remain in the circulation as shown here, in that it enters the medium reservoir 2, from where it is extracted for a repeated continuous perfusion of the transplant 11. It can also be completely removed from the circulation. The transplant 11 is then cultivated by means of a batch or fed-batch process respectively.

The integration of a second chamber in the invention, shown here as being underneath the transplant 11, especially shows its positive feature in a targeted approach flowing of the biological construct. If the media flow from media reservoir 2 is switched to the lower chamber by means of the 3-way valve 6, an induced upwards-oriented perfusion of the transplant 11 takes place whilst the lower discharge is closed due to the medium only being able to leave the reactor chamber via the upper discharge.

Analogous to this schema, a switching over of the 3-way valve 6 results in a transplant through-flow from the upper to the lower chamber through the construct 11. The arrangement described here results not only in a complete perfusion, but also in an additional cell stimulation via an induced shearing force which is asserted on the cells and can be adjusted via the volume flow of the circulation pump 5. A partial or complete opening of the 3-way valve 6 is also possible in order to achieve a faster medium exchange in the bioreactor 1.

### EXAMPLE 9

Schema Showing the Fixation of the Transplant 11 in the Bioreactor 1

FIG. 9 are schemas showing the fixation of the transplant 11 in bioreactor 1, irrespective of whether it is the single-chamber or the-double-chamber version.

FIG. 9a shows the transplant which is to be stimulated 11 which is medially fixated above the transparent glass 17 in the single-chamber bioreactor 1. With a minimum of 3 of these fixation walls 28, a horizontal movement of the transplant 11 on the reactor floor as a result of the incoming medium flow should avoided in order to enable an optimal perfusion and pressure stimulation. These biocompatible fixation walls 28 which are inserted in the reactor 1 must be of a height which is lower than the pressure amplitude which is to be applied to the transplant 11.

FIG. 9b shows the use of at least 3 of these fixation walls 28 in a double-chamber bioreactor in order to achieve a horizontal fixation of the transplant 11 in diverse flow situations, thereby enabling an ideal vertical perfusion and a mechanical pressure application to take place.

### EXAMPLE 10

Magnet Systems for Controlling the Mini Actuator 14

FIG. 10 show characteristic devices and apparatus arrangement (shown in a single-chamber bioreactor) for a contactless controllable stimulation process for the mini actuator 14 on the transplant 11.

FIG. 10 (magnetic control effect—magnet attraction) shows the characteristic arrangement and principle of the contactless controlling of the magnetic mini actuator 14 in the bioreactor 1 for the pressure deformation of the transplant 11. The alignment of the permanent magnets in the mini actuator 14 is carried out in accordance with the predominant magnetic field direction which is generated by externally situated control magnets 15. These control magnets 15 which is at least a permanent magnet or at least a coil generates a defined (electro-)magnetic field which protrudes into the complete bioreactor chamber 1 with its field lines and triggers a field direction-related movement of the mini actuator 14 pressure stamp. In the example shown in FIG. 10a, the control magnets 15 show the principle of the magnet attraction, taking an arrangement from above as an example.

In the example execution shown in FIG. 10b (magnetic control effect—pushing off of the magnet) the pushing off of the magnet represents the second magnetic control effect between the magnetic control system 15 and the mini actuator 14. A changing of the magnetic field direction of the control magnets 15 results in an alteration of the direction of movement of the mini actuator 14 which is now steered in the direction of the transplant 11 with an upwards orientation. By increasing the performance or magnetic flow density from the control magnets 15, the pressure load applied to the transplant-11 is increased until it reaches the target value of the in-vivo adaptive stimulation.

The FIGS. 10c-10e show arrangements of control elements which can be used to steer the mini actuator 14 in a self-contained bioreactor 1 in a cyclic manner and with a high frequency.

FIG. 10c (controlling the mini actuator 14 by means of a control magnet guide plate) shows a form of execution of a permanent magnetic control system. In this magnetic field version, an arrangement of numerous permanent magnets 32 of various sizes and with various polarities and therefore field strengths and directions works on a linear-controlled guide plate 31, this being shown here as being positioned above the reactor prototype as an example.

Hereby, a linear motor 29 drives a guide rail 30 with the permanent magnets 32 which are situated in the magnet holder 31. This mobile phase of the magnet system renders a movement of the bioreactor 1 unnecessary.

The control system in FIG. 10d (controlling the mini actuator 14 by means of rotating permanent magnets) is also based on a controlling of the magnetic pressure stamp 14 by means of an arrangement of permanent magnets on a rotating disk.

Hereby, a servomotor 29 drives a magnet holder 31 containing adapted permanent magnets 32 with alternating polarities. This rotating magnet holder can include four alternating polarized magnets 32 and as a result they bring about a full rotation of two complete pressure applications to the transplant 11. The combination of this occupancy of the rotating discs with magnets and the rotary speed of the servomotor 29 produce a magnetic field alteration with a greater frequency and therefore a highly dynamic stimulation pattern on the transplant 11. The front view makes both of the magnet effects on the rotation system clear, taking two bioreactors 1 as an example. The form of execution of this arrangement is suitable for numerous bioreactors 1 as long as these can be exactly positioned above or underneath the centre of the control magnet.

FIG. 10e (controlling the mini actuator 14 by means of an iron core coil 35) shows a magnet device based on a coil arrangement.

This magnet coil system works with an induction coil 35, which is fixated above the bioreactor 1 with generation of a defined electromagnetic field which can be invariably adjusted via the supplied electrical power, thereby enabling the mini actuator 14 to be positioned anywhere in the bioreactor carcass. A pole reversal of the direction of current results in a reversal of the existing field direction and the electromagnetic effect. The used iron core coils 35 generates its electrical field vertical to the coil winding and has both an attracting and push off effect on the static permanent magnets of the mini actuator 14.

An automated station of this system comprises a powerful coil 35 with a low heat generation and a connected adjustable transformer, the capacity of which being monitored by a multimeter measuring device. Furthermore, the use of a microcontroller triggers a relay which switches the current in the required direction, ensuring the required effect of an intermittent pressure application to the cell construct.

### EXAMPLE 11

Stimulation Schema in a Double-Chamber Reactor

FIGS. 11 show the complete stimulation schema of the novel GMP-conform bioreactor 1. Hereby, the mechanical pressure stimulation, perfusion and the shearing force-induced flow takes place parallel in the three-dimensional transplant.

In FIG. 11a (perfusion stimulation without mechanical load), a stimulation of the cell construct 11 only takes place via a targeted approach flow of the media, resulting in a construct perfusion with an assertion of the shearing force in a μPa-range. This process example shows a continuous feeding of culture media in two feeds 19 so that a supply is provided to each of the reactor chambers initially adjusts itself to a concentration equalization in transplant 11 and thereafter generates an upper and a lower perfusion zone on the construct in relation to the selected volume flows. This used medium leaves the reactor chamber via two additional discharges 19. No pressure is applied during this flow stimulation as the pressure stamp 14 is held in a higher position in the bioreactor 1 by the control magnet system 15.

In FIG. 11b (perfusion stimulation and stamp application) shows the second step which is a multiple stimulation of replacement tissue materials 11 in the bioreactor 1. As is shown in this example, the flow conditions are initially modified. Via the 3-way valve 6, the culture medium flow is only fed into the lower reactor chamber, from where it is perfused through the transplant 11, the material exchange induced and it can then leave the upper reactor chamber via a discharge. By reversing the poles of the control magnet system, this being an iron core coil 35 with a low power induction in this case, the magnetic mini actuator 14 is placed on the cylindrical replacement tissue 11, for example. This stamp placement with a 0% construct deformation marks a return point of the mini actuator 14 with a dynamically high-frequency deformation of the cell matrix 11.

In the next step of the stimulation process, the magnetic field strength generated by the coil 35 is increased as shown in FIG. 11c (perfusion stimulation and mechanical load). The result of this increased magnetic flow density is an increased compression of the transplant 11 to the required target deformation which preferably imitates process which is similar to in-vivo processes. After this pressure stimulation has been carried out, a change can be made between cell stimulation and stamp application in an intermittent manner.

A static compression of the replacement material is also possible with the cited apparatus and the described process. During this mechanical load, a targeted construct perfusion can be inserted through the carrier matrix which supplies the cells with the required nutrients and metabolites removed which are especially exchanged, e.g. during the proliferation and differentiation (extracellular matrix synthesis).

After the pressure load protocol has been worked off, one returns the stamp device back to the starting position, continues to perfuse the cell culture continuously, for example and removes the transplant 11 if the extracellular matrix has been sufficiently synthesized, for example.

1-57. (canceled)

58. A process for the cultivation and stimulation of three-dimensional, vital and mechanically-resistant cell transplants in a GMP-conform bioreactor, the process which comprises the following steps:
a) taking explant cells from an organism and preparing the explant cells for bioreactor cultivation and mixing a carrier matrix comprising commercially-available biocompatible, absorbable or autologous or homologous materials to form a cell matrix suspension;
b) placing the cell matrix suspension in a, optionally foiled, seeding piston having a cross-section adapted to a later transplant, and hardening or polymerizing therein;
c) optionally loading, with minimum pressure by way of an exactly fitting, inert stamp that is optionally structured or foiled;
d) inserting the seeding piston with the transplant into a chamber space of the bioreactor body;
e) medially placing the transplant from the seeding piston on the floor of the bioreactor, removing the seeding piston, and closing the bioreactor;
f) further cultivating the transplant by feeding a perfusion flow, and during the cultivation phase loading the transplant to a load pressure on a surface thereof opposite the floor of the bioreactor; and
g) after a completion of the cultivation, removing the replacement tissue material for further use.

59. The process according to claim 58, which comprises subjecting the transplant to a load with a stamp applying pressure.

60. The process according to claim 58, which comprises selectively controlling a blending in the bioreactor chamber due to the perfusion flow and the load pressure with regard to time and quantity or density in relation to the cultivation conditions.

61. The process according to claim 58, wherein the transplant has conditioned cultivating medium flow through it at intervals and is subjected to loading in cycles with the load pressure.

62. The process according to claim 58, which comprises subjecting the transplant to the load pressure during the perfusion flow.

63. The process according to claim 58, which comprises stimulating the transplant in accordance with its use with with static pressure loads, with in-vivo-simulating pressure loads or construct deformations, or with continuous-load intermittent or dynamic pressure forces.

64. The process according to claim 58, which comprises applying the mechanical load with a frequency exceeding 0.1 Hz.

65. The process according to claim 58, which comprises subjecting the transplant to mechanical pressure stimulation in the form of a symmetrical or asymmetrical half cosine or sine wave.

66. The process according to claim 59, which comprises providing a pressure stamp with magnetic material and moving the pressure stamp longitudinal to the surface of the transplant in the bioreactor by a magnetic field or an electro-magnetic field generated outside the bioreactor.

67. The process according to claim 66, which comprises generating the magnetic field with at least one permanent magnet.

68. The process according to claim 66, which comprises positioning at least two permanent magnets with alternating polarity on a mobile holder above the bioreactor and driving the holder by a servomotor in a cyclic manner, to thereby change a position thereof resulting in the pressure stamp applying pressure to the transplant alternately.

69. The process according to claim 66, wherein the coil of an electromagnet alters, at a high frequency, a current direction and a voltage and therefore a field direction and a magnetic flow density through the bioreactor, whereby the pressure stamp applies pressure to the transplant alternately.

70. A bioreactor for cultivating and stimulating three-dimensional, vital and mechanically resistant cell transplants in an GMP-conform bioreactor, the bioreactor comprising:
a basic bioreactor body with a reactor lock connected thereto in a pressure-proof and sterile manner to define therein at least one reactor chamber;
said reactor chamber having a support surface for a transplant formed therein and a mini actuator disposed therein; and
at least two hose coupling connections for feeding and discharging of a medium or for gassing said reactor chamber.

71. The device according to claim 70, wherein the reactor is a single-chamber bioreactor and the cell culture constructs are directly or indirectly cultivated and stimulated on a bioreactor floor of the single-chamber bioreactor.

72. The device according to claim 70, wherein the reactor is a double-chamber bioreactor and the cell culture constructs are directly or indirectly, at least partially positioned on a floor of an upper reaction chamber of the double-chamber bioreactor for cultivation and stimulation, while the transplant is located in a second reactor chamber.

73. The device according to claim 72, which comprises a transplant insert on the floor of the upper reactor chamber for receiving therein the cell constructs.

74. The device according to claim 70, wherein said basic reactor body is a cylinder-shaped corpus closed from above with said reactor lock.

75. The device according to claim 70, wherein said reactor lock is one or more reactor lock units connected to said bioreactor by one threaded joint and at least one conical nipple such that a threaded joint is either created between the reactor lock and the container (1) by a female thread in the container and a male thread in the reactor lock working together or the threaded joint is created between the reactor lock and the container in that a male thread in the container and a female thread in the reactor lock work together.

76. The device according to claim 70, wherein said reactor lock is a cover equipped with biosensors and/or measuring heads.

77. The device according to claim 76, wherein said cover is equipped with a sample taking section.

78. The device according to claim 71, wherein said basic body of said single-chamber bioreactor has at least two each of a feed and discharge borehole for hose coupling connections.

79. The device according to claim 78, wherein said feed connections and discharge connections communicating with said bioreactor chamber are fitted with a 3-way valve or a 4-way valve with a return function.

80. The device according to claim 79, wherein at least one of said discharge connections is formed with a sample taking section.

81. The device according to claim 72, wherein said basic body of said double-chamber bioreactor has at least two boreholes for hose coupling connections.

82. The device according to claim 81, wherein at least one hose coupling connection is integrated in a lower reaction chamber and at least one is integrated in an upper reaction chamber.

83. The device according to claim 82, wherein said hose coupling connections communicating with said bioreactor chamber are fitted with a 3-way valve or a 4-way valve with a return function.

84. The device according to claim 83, wherein at least one of said discharge connections is formed with a sample taking section.

85. The device according to claim 70, wherein the bioreactor has a reactor floor of a completely or partially transparent material for monitoring the transplant manufacture.

86. The device according to claim 70, which comprises a foil, a fleece or a membrane of an antistatic or inert material disposed above the reactor floor of the bioreactor for the positioning of the transplant.

87. The device according to claim 86, wherein the material for positioning the transplant is wide-meshed and light, fluid and gas permeable.

88. The device according to claim 72, wherein the upper reactor chamber of said double-chamber bioreactor has an area corresponding to a transplant area while the dimensions of the lower chamber are less than those of the transplant so that if a cell culture is placed medially, the construct is mainly positioned underneath the lower chamber and lies partially on the reactor floor of the upper chamber.

89. The device according to claim 88, wherein the space underneath the reactor chamber is filled out by a flat plate of a biologically inert, light-permeating, wide-pored material, said plate being flush with the floor of the upper reactor chamber.

90. The device according to claim 89, wherein said plate is formed of a porous sinter material.

91. The device according to claim 89, wherein a foil, fleece or membrane of an antistatic or inert material for positioning the transplant disposed above the lower reactor chamber that is filled out by said plate on the reactor floor of the upper reactor chamber of said double-chamber bioreactor.

92. The device according to claim 91, wherein said material is wide-meshed and permeable to light, fluid and gas.

93. The device according to claim 89, wherein the components underneath the transplant in said double-chamber bioreactor, including the transparent plate, the lower chamber with the inserted porous material and a wide-meshed membrane have an overall height not exceeding a focal distance of conventional microscopes and camera objectives.

94. The device according to claim 70, wherein said mini actuator comprises a magnetic piston-type actuator disposed in said bioreactor and movalby through said bioreactor under control of one or more externally disposed control and steering magnets.

95. The device according to claim 71, wherein said mini actuator in said single-chamber bioreactor is situated above said membrane and the transplant in said bioreactor chamber.

96. The device according to claim 72, wherein said mini actuator in said double-chamber bioreactor is situated in the upper reactor space above a porous material, above a membrane and the transplant.

97. The device according to claim 94, wherein said magnetic mini actuator is formed of a magnetic core encapsulated in a biologically inert enveloping body.

98. The device according to claim 97, wherein said magnetic core is oriented to cause a field generated thereby between the poles runs vertically to the transplant, with a magnetic north pole of said mini actuator oriented in an upward direction.

99. The device according to claim 97, wherein said enveloping body is a biocompatible enveloping body surrounding said core and having an external form matching a form of the reactor chamber of the bioreactor.

100. The device according to claim 97, wherein a complete height of said enveloping body is such that a placement of said mini actuator in the reactor space results in a vertically-oriented guiding of a pressure stamp of said mini actuator towards the transplant.

101. The device according to claim 97, wherein said mini-actuator is a piston-shaped mini actuator comprising a plurality of enveloping body cylinders, with one of said enveloping body cylinders containing the encapsulated permanent magnet and an additional cylinder serving the stamp impression, and a bridge connection joining the spatially separated cylinders.

102. The device according to claim 97, wherein a planar stamp surface on an underside of said mini actuator formed by said enveloping body runs vertical to a guide direction in the bioreactor space.

103. The device according to claim 97, wherein a stamp surface of said mini actuator has organotypical negative forms embossed thereon.

104. The device according to claim 97, wherein a planar or formed stamp surface is embossed with a grid structure for increasing a stamp surface.

105. The device according to claim 97, wherein said enveloping body of said mini actuator is formed with drill holes and/or flow channels guaranteeing a continued exact vertical guiding of said mini actuator at at least three locations of the periphery.

106. The device according to claim 97, wherein the stamp surface of the mini actuator is fitted with at least one nosepiece which slides into its exactly fitting integrated guide rail in the bioreactor body.

107. The device according to claim 97, wherein a control and steering magnet disposed outside the bioreactor brings about an oriented movement of said mini actuator with the magnetic or electromagnetic field which it generates with the north pole of the permanent magnet that is oriented upwards.

108. The device according to claim 97, wherein a control and steering magnet is medially situated in a vertical axis to the pressure stamp, preferably above the pressure stamp and moves upwards and downwards in relation to the polarity of the mini actuator, resulting in an alteration of the pressure applied to the transplant.

109. The device according to claim 94, wherein said control and steering magnet comprises two permanent magnets with different vertical magnetic pole directions which are inserted in a rectangular shaped magnet holder and moved to their horizontal position above the bioreactor in a cyclic manner by means of a servomotor and a guide rail.

110. The device according to claim 94, wherein said control and steering magnet comprises a minimum of two permanent magnets with different vertical magnetic pole directions, said permanent magnets being in a disk-shaped magnet holder and moved over the bioreactor in a cyclic manner as a result of the rotation drive of a servomotor.

111. The device according to claim 110, wherein the bioreactors which are firmly fixed in their horizontal position approach the permanent magnets via a vertical movement of the magnet holder by way of a step motor, in order to increase a field effect and generate an application of higher pressures on the transplant.

112. The device according to claim 111, wherein at least two bioreactors are so arranged in a station that their mini actuators are driven by just one permanent magnetic control system in a contactless manner.

113. The device according to claim 94, wherein said control and steering magnet is an electromagnet with at least one induction coil with an infinitely variable field.

114. The device according to claim 113, wherein said induction coil is highly frequently triggered by frequencies which can be altered in order to generate high-dynamic magnetic field alterations and mini actuator movement on the transplant.

115. The device according to claim 70, which comprises a seeding piston with an inside diameter corresponding to an outside diameter of the transplants is disposed on a moving sliding plate for injecting the cells and the carrier matrix.

116. The device according to claim 115, wherein said moving sliding plate and the inside of the seeding piston are coated by an inert membrane, foil or polymer fleece.

117. The device according to claim 115, wherein an exactly fitting stamp with a planar stamp surface or an organotypical negative form in the seeding piston is lightly applied to a cell suspension.

118. The device according to claim 115, wherein an outside diameter of said seeding piston exactly matches an inside diameter of said bioreactor.

119. The device according to claim 70, wherein at least three fixation walls are integrated in the reactor floor of said bioreactor, said fixation walls having dimensions for accommodating a transplant insert and to not impair a pressure compression of the transplant.
